(19) United States
(12) Patent Application Publication
(10) Pub. No.: US 2026/0364268 A1
(43) Pub. Date: Nov. 19, 2026

(54) MEDICAL PUNCTURE NEEDLE

(71) Applicant: TERUMO KABUSHIKI KAISHA, Tokyo (JP)

(72) Inventor: Kentaro NAKAJIMA, Muntinlupa City (PH)

(73) Assignee: TERUMO KABUSHIKI KAISHA, Tokyo (JP)

(21) Appl. No.: 19/732,768

(22) Filed: Jul. 7, 2026

Related U.S. Application Data
(63) Continuation of application No. PCT/JP2024/025127, filed on Jul. 11, 2024.

(30) Foreign Application Priority Data
Jan. 25, 2024 (JP) 2024-009749

(51) Int. Cl. A61M 5/32; A61M 25/00

(52) U.S. Cl. CPC A61M 5/3202; A61M 25/0082

(57) ABSTRACT

A medical puncture needle includes: a needle unit including: a needle member including a needle that has a lumen, a holder holding a proximal end of the needle member, and a patch having a first surface configured to be attached to a body surface of a living body, and a second surface opposite to the first surface, at least a part of the second surface being connected to the holder; and a needle protection member that covers an outer periphery of the needle and is removable from the needle unit when the needle is used, the needle protection member including: a cover portion extending in an axial direction to cover the outer periphery of the needle. When viewed from an axial direction of the needle, the opening is larger than the needle.

### CROSS-REFERENCE TO RELATED APPLICATIONS

This is a bypass continuation of PCT Application No. PCT/JP2024/025127, filed on Jul. 11, 2024, which claims priority to Japanese Patent Application No. 2024-009749, filed on Jan. 25, 2024. The entire contents of these applications are hereby incorporated by reference herein.

### BACKGROUND

The present disclosure relates to a medical puncture needle.

WO 2023/188851 A discloses a medicinal liquid administration device including a needle. The needle of the medicinal liquid administration device is a multiple needle including an outer needle (catheter) and an inner needle, the outer needle being capable of puncturing the skin of a living body and being indwelled. In an initial state before use of the medicinal liquid administration device, the needle is covered with a needle protection member. In an assembly process of the medicinal liquid administration device, a tubular needle protection member is attached to an outer periphery of the needle.

### SUMMARY

In the assembly process of the medicinal liquid administration device, the needle protection member may contact the distal end of the needle and damage the distal end.

An object of certain embodiments of the present disclosure is to solve the above-described problem.
(1) An aspect of the present disclosure is a medical puncture needle including a needle unit including a needle having a lumen, and a needle protection member that protects the needle by covering an outer periphery of the needle and that is removable from the needle unit when the needle is used, wherein the needle unit includes a needle member having the needle, a holder holding a proximal end of the needle member, and a patch having a first surface attached to a body surface of a living body punctured by the needle and a second surface opposite to the first surface, at least a part of the second surface being connected to the holder, the needle protection member includes a cover portion having a tubular shape having an accommodation space in which the needle is accommodated and extending in an axial direction to cover the outer periphery of the needle, and an opening provided at a proximal end of the cover portion and continuous with the accommodation space, when viewed from an axial direction of the needle, the opening is larger than the needle, and in a state in which the needle is accommodated in the cover portion, the cover portion and the needle are out of contact with each other.

According to this medical puncture needle, when the needle protection member is attached to the needle unit in the assembly process, contact of the distal end of the needle with the opening of the cover portion can be avoided, and therefore damage to the distal end of the needle due to contact with the proximal end of the cover portion can be suppressed. In addition, in a state in which the needle is accommodated in the needle protection member, the needle protection member and the needle are out of contact with each other, and therefore damage to the distal end of the needle due to contact with the cover portion can be suppressed.
(2) In the medical puncture needle according to (1) above, the needle may be a catheter having flexibility, and the medical puncture needle may include an inner needle inserted through the catheter.

This configuration can effectively protect the distal end of the catheter having flexibility.
(3) In the medical puncture needle according to (1) or (2) above, the needle protection member may be supported by the holder.

This configuration enables support of the needle protection member with a simple configuration by supporting the needle protection member with the holder.
(4) In the medical puncture needle according to (1) above, the needle member may be an outer needle member, the needle unit may include an inner needle member having an inner needle inserted into the lumen of the needle and a needle holding part holding a proximal end of the inner needle, and the inner needle member may be detachably attached to the outer needle member.

This configuration can suppress damage to the distal end of the needle forming an outer side of the double needle in an assembly process of the medical puncture needle including the needle unit having the double needle.
(5) In the medical puncture needle according to (4) above, the needle protection member may be supported by the holder or the needle holding part.

This configuration enables support of the needle protection member in a state in which the needle is accommodated, with a simple configuration.
(6) In the medical puncture needle according to any one of (1) to (5) above, the needle protection member may have a patch accommodating portion that accommodates at least a part of the patch.

This configuration can effectively suppress deformation, damage, and the like of the patch before use by protecting the needle with the needle protection member and accommodating the patch in the patch accommodating portion.
(7) In the medical puncture needle according to (6) above, the needle protection member may hold the distal end of the needle and the patch in a state in which the distal end of the needle and the patch are spaced apart from each other.

This configuration can keep the distal end of the needle clean because contact of the distal end of the needle with the patch is prevented by the needle protection member.
(8) In the medical puncture needle according to (6) above, the patch accommodating portion may be a slit portion provided in the cover portion of the needle protection member and intersecting an inner wall of the cover portion.

This configuration enables, in the needle protection member, accommodation of the needle in the cover portion and accommodation of the patch by the slit portion to be performed simultaneously.
(9) In the medical puncture needle according to (8) above, the cover portion may have a first region including a distal end portion of the cover portion and a second region including at least a part of a proximal end portion of the cover portion and separated from the first region by the slit portion, and the needle protection member may have a connection portion connecting the first region and the second region on an outer surface of the cover portion.

This configuration enables a large accommodation range of the patch to be secured by the slit portion, and enables a suitable size of the patch to be secured.
(10) In the medical puncture needle according to (9) above, the slit portion may extend from the opening to the connection portion.

This configuration enables, by enlarging a range of the slit portion, a larger accommodation range of the patch to be secured and a large patch to be accommodated.

According to the present invention, a medical puncture needle includes a needle protection member that protects a needle by covering an outer periphery of the needle and that is removable from a needle unit when the needle is used, and the needle protection member includes a tubular cover portion that covers the outer periphery of the needle. In a state in which the needle is accommodated in the cover portion, the cover portion and the needle are provided out of contact with each other. As a result, when the needle protection member is attached to the needle unit in the assembly process of the medical puncture needle, contact of the distal end of the needle with the opening of the cover portion can be avoided. Therefore, damage to the distal end of the needle due to contact with the proximal end of the cover portion can be suppressed. In addition, in the state in which the needle is accommodated in the needle protection member, the needle protection member and the needle are out of contact with each other, and therefore damage to the distal end of the needle due to contact with the cover portion can be suppressed.

### BRIEF DESCRIPTION OF THE DRAWINGS

FIG. 1 is an overall plan view of a medicinal liquid administration device including the medical puncture needle according to a first embodiment of the present invention.

FIG. 2 is an overall cross-sectional view of the medicinal liquid administration device illustrated in FIG. 1.

FIG. 3 is an enlarged cross-sectional view illustrating a vicinity of the medical puncture needle in the medicinal liquid administration device.

FIG. 4 is a cross-sectional view taken along line IV-IV in FIG. 1.

FIG. 5 is an explanatory view illustrating a state in which a device main body is attached to the medicinal liquid administration device of FIG. 1.

FIG. 6 is an explanatory view illustrating a state in which the needle protection member is removed from the medicinal liquid administration device illustrated in FIG. 2.

FIG. 7 is an external perspective view of the needle protection member.

FIG. 8 is an explanatory view of the medical puncture needle as viewed from a proximal direction.

FIG. 9 is an exploded cross-sectional view illustrating a state in which the needle protection member is removed from the needle unit.

FIG. 10 is a perspective cross-sectional view illustrating a distal end of the medicinal liquid administration device.

FIG. 11 is an explanatory view illustrating puncture by the needle of the medicinal liquid administration device.

FIG. 12 is a plan view of a medicinal liquid administration device including a medical puncture needle according to a modification.

FIG. 13 is a perspective cross-sectional view illustrating a distal end of the medicinal liquid administration device of FIG. 12.

FIG. 14 is an overall plan view of a medicinal liquid administration device including the medical puncture needle according to a second embodiment of the present invention.

FIG. 15 is a cross-sectional view taken along line XV-XV in FIG. 14.

FIG. 16 is a cross-sectional view taken along line XVI-XVI in FIG. 15.

### DETAILED DESCRIPTION

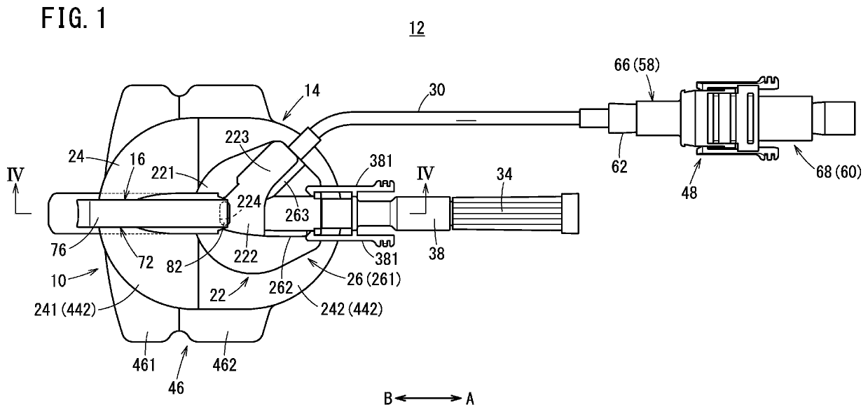

As illustrated in FIG. 1, a medical puncture needle 10 according to the present embodiment is used in a medicinal liquid administration device 12.

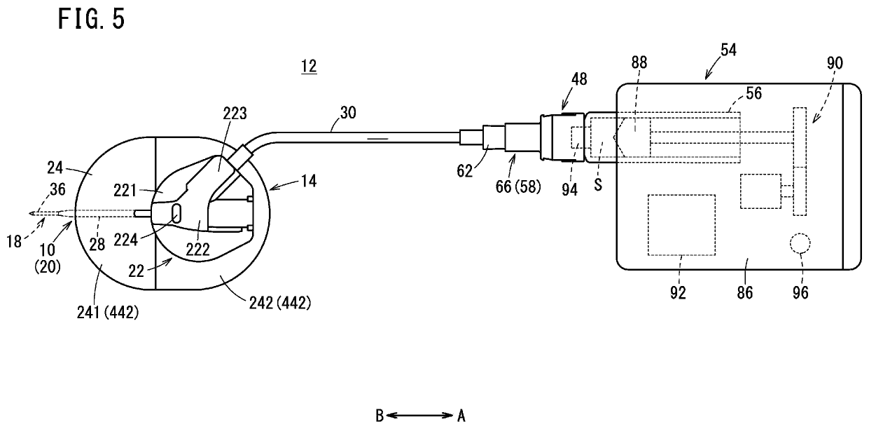

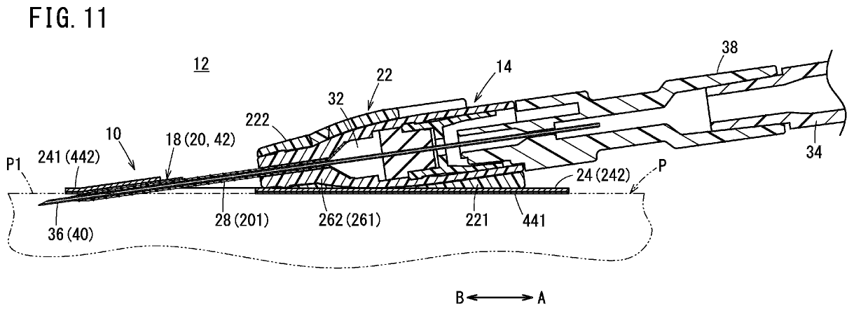

The medicinal liquid administration device 12 is configured to be indwelled through a body surface P1 (see FIG. 11), such as an abdomen, of a user P (living body), and automatically administer a medicinal liquid S (see FIG. 5) into the body (inside the living body). For example, the medicinal liquid administration device 12 is used when administering the medicinal liquid S to the user P at a timing when a predetermined time has elapsed after performing a medical procedure on the user P, or when gradually administering the medicinal liquid S over time. The medicinal liquid S administered by the medicinal liquid administration device 12 is not particularly limited. Examples of the medicinal liquid S include liquid pharmaceuticals such as antibody drugs, anticancer agents, chemotherapeutic agents, anesthetics, antibiotics, insulin, blood products, and nutritional agents.

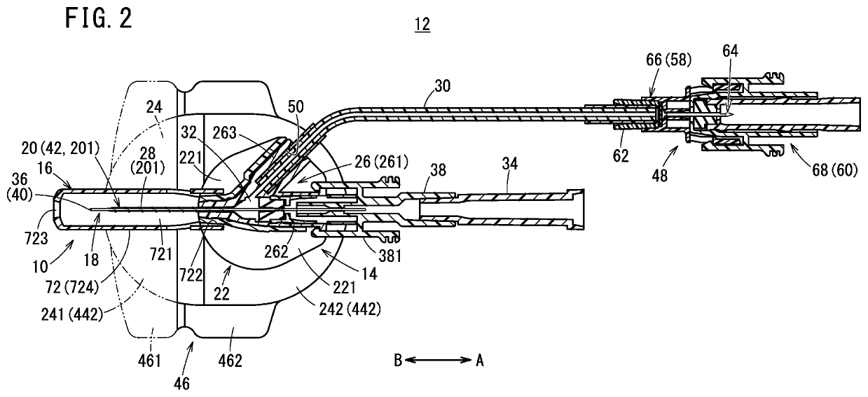

As illustrated in FIG. 2, the medicinal liquid administration device 12 includes the medical puncture needle 10. The medical puncture needle 10 includes a needle unit 14 and a needle protection member 16 (which may also be referred to as a protection member 16) that covers an outer periphery of the needle 18 of the needle unit 14 and is removable from the needle unit 14.

The needle unit 14 includes a needle member 20 having the needle 18, a holder 22 holding a proximal end of the needle member 20, and a patch 24 connected to the holder 22.

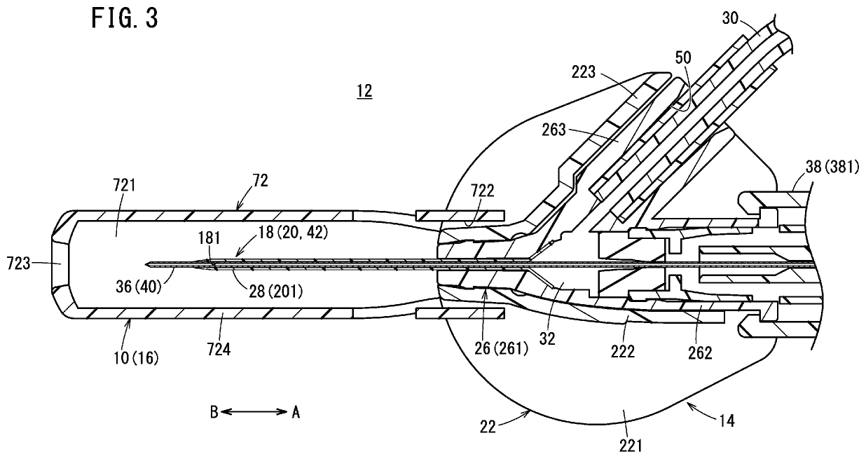

As illustrated in FIG. 3, the needle member 20 (which may also be referred to as a catheter member 20) is an outer needle member 201. The outer needle member 201 includes the needle 18 and a catheter hub 26 holding a proximal end of the needle 18. The needle 18 is a catheter 28 having flexibility and a lumen 181. The catheter 28 is inserted through the skin of the user P (see FIG. 11) and is indwelled in the body (subcutaneously).

The catheter 28 is a tubular body including the lumen 181 through which the medicinal liquid S can flow. The lumen 181 of the catheter 28 is in communication with a distal opening of the catheter 28. The distal end of the catheter 28 projects obliquely downward from a lower surface of the catheter hub 26 (a surface facing the body surface P1) toward a distal direction (the direction of arrow B) (see FIG. 11).

The catheter hub 26 is a needle hub 261. The needle hub 261 includes a hub main body 262 supporting a proximal end of the catheter 28, and a side port 263 projecting from a side portion of the hub main body 262 and connected to a distal end of a tube 30. The hub main body 262 extends linearly from a distal end toward a proximal end and has a space 32 inside the hub main body 262. The space 32 is in communication with the lumen 181 of the catheter 28 and the tube 30 connected to the side port 263. As illustrated in FIG. 2, a needle holding part 38 to which a gripping member 34 is bonded is detachably attached to a proximal end of the hub main body 262. The proximal end of the catheter 28 is fixed to the needle hub 261 by caulking a caulking pin (not illustrated).

A distal end of the needle holding part 38 has pressing portions 381. The pressing portions 381 are provided on an outer peripheral surface of the needle holding part 38, and one pair of the pressing portions 381 is provided in a width direction orthogonal to an axial direction of the needle holding part 38. When the needle holding part 38 is pulled out from the catheter hub 26 in the proximal direction, the user P presses proximal end portions of the pressing portions 381 inward in the width direction. By pressing the proximal end portions of the pressing portions 381, distal end portions of the pressing portions 381 tilt outward in the width direction, and engagement of the needle holding part 38 with the catheter hub 26 can be released.

As illustrated in FIG. 3, the needle unit 14 further includes an inner needle member 40 having an inner needle 36 inserted into the lumen 181 of the needle 18 (catheter 28), and the needle holding part 38 holding a proximal end of the inner needle 36. The inner needle 36 is made of a metallic material. A part of the inner needle 36 passes through an interior of the catheter 28. A distal side of the inner needle 36 is inserted through the lumen 181 of the catheter 28. A proximal end of the inner needle 36 is inserted through an interior of the needle hub 261. The inner needle 36 being inserted through the catheter 28 forms a double needle 42 overlapped in a radial direction. By inserting the catheter 28 into the body from the body surface P1 of the user P (see FIG. 11) and indwelling the catheter 28, an introduction portion for the medicinal liquid S is formed. After puncturing the body with the double needle 42 including the catheter 28 and the inner needle 36, the user P indwells the catheter 28 in the body of the user P by pulling the inner needle 36 out from the catheter 28 in the punctured state. That is, the inner needle member 40 is attached to the outer needle member 201 so as to be removable in the proximal direction.

As illustrated in FIG. 1, the holder 22 has a base portion 221 formed in a flat plate shape and to which the patch 24 is fixed, and a hub holding part 222 and a port holding part 223 that are provided on an upper part of the base portion 221 and hold the needle hub 261.

The base portion 221 is formed in a substantially circular shape in a top view. The hub holding part 222 and the port holding part 223 are provided on a surface of the base portion 221.

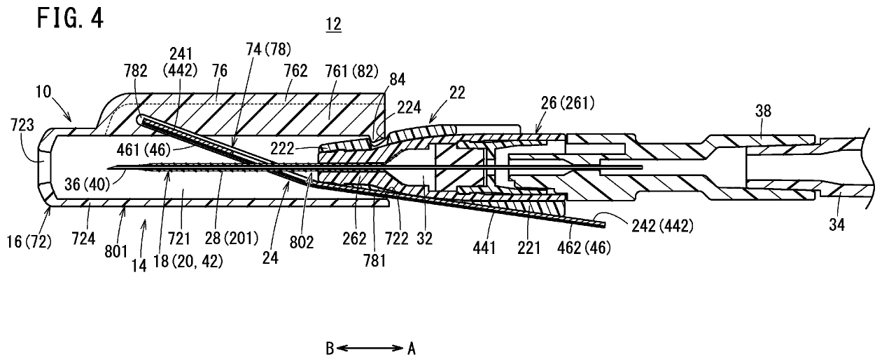

As illustrated in FIG. 3, the hub holding part 222 holds the hub main body 262 of the needle hub 261. The hub holding part 222 is formed in a tubular shape and is disposed at a center of the base portion 221. The hub holding part 222 extends along an extending direction of the catheter 28. A distal end of the hub holding part 222 is disposed so as to be inclined toward the base portion 221 (see FIG. 4). As illustrated in FIG. 5, a hole 224 is provided at a connection site between the hub holding part 222 and the port holding part 223. As illustrated in FIG. 4, when the needle protection member 16 is attached so as to cover an outer periphery of the catheter 28, a proximal end portion of a second region 802 of the needle protection member 16 covers a distal end portion of the hub holding part 222.

As illustrated in FIG. 2, the patch 24 has a first patch portion 241 through which the catheter 28 is inserted, and a second patch portion 242 that is disposed at a proximal end of the first patch portion 241 and to which the holder 22 is fixed. As illustrated in FIG. 4, the patch 24 has a first surface 441 attached to the body surface P1 of a living body (see FIG. 11), and a second surface 442 opposite to the first surface 441. Each of the first and second surfaces 441 and 442 is formed over the first and second patch portions 241 and 242. At least a part of the second surface 442 of the patch 24 is fixed to a back surface of the base portion 221 of the holder 22. The second surface 442 is covered with a release sheet 46. The release sheet 46 has a first sheet portion 461 covering the first surface 441 of the first patch portion 241, and a second sheet portion 462 covering the first surface 441 of the second patch portion 242.

The medicinal liquid administration device 12 further includes a tube 30 held by the needle hub 261 and allowing the medicinal liquid S (see FIG. 5) to flow, and a connector 48 provided at a proximal end portion of the tube 30.

The tube 30 is formed from a flexible tube through which the medicinal liquid S can flow inside, and a distal end portion of the tube 30 is fixed to a connection port 50 of the needle hub 261. As a result, the tube 30 communicates with the space 32 of the needle hub 261 through the connection port 50.

The connector 48 is selectively connectable to a priming liquid delivery tool (not illustrated) capable of delivering priming liquid (physiological saline) filled therein from a distal end portion, and to a device main body 54 (see FIG. 5) capable of delivering the medicinal liquid S filled therein.

The connector 48 has a first connection part 58 connectable to a prefilled syringe 56 (see FIG. 5) of the device main body 54, a second connection part 60 connectable to a priming liquid delivery tool (not illustrated) capable of delivering priming liquid, and a tube holder 62 that holds a proximal end of the tube 30 and is attached to the first connection part 58.

The first connection part 58 has a first connection member 66 connected to the proximal end portion of the tube 30 via the tube holder 62. A communication state between an interior of the first connection member 66 and the tube 30 is switched by a valve body not illustrated.

As illustrated in FIG. 1, the second connection part 60 has a second connection member 68 detachably attached to a proximal end portion of the first connection member 66. A distal end portion of a priming liquid delivery tool not illustrated is connectable to the second connection member 68.

The tube holder 62 has a bottomed cylindrical shape having an open distal end. The tube holder 62 is fitted and fixed by being inserted into a holder hole (not illustrated) of the first connection member 66. The proximal end portion of the tube 30 is inserted and fixed inside the tube holder 62.

As illustrated in FIG. 2, when the second connection member 68 is connected to the first connection member 66, an interior of the first connection member 66 and an interior of the second connection member 68 are connected and brought into communication by a hollow needle 64.

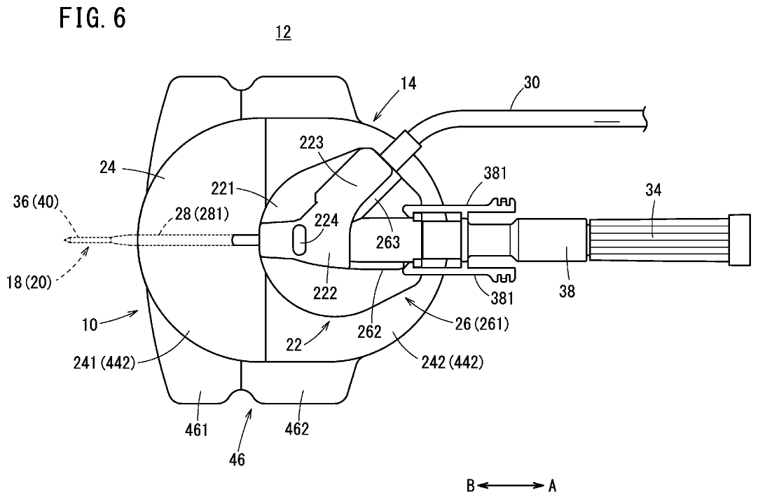

As illustrated in FIG. 3, the needle protection member 16 is detachably disposed so as to cover an outer periphery of the catheter 28. In an initial state before use in which puncture by the inner needle 36 and the catheter 28 (double needle 42) is performed, the needle protection member 16 is attached so as to cover the outer periphery of the catheter 28. By covering the outer periphery and the distal end of the double needle 42 including the catheter 28 with the needle protection member 16, accidental puncture by the inner needle 36 before use is prevented. When puncture by the double needle 42 is performed, the needle protection member 16 is removed from the needle unit 14 (see FIGS. 5 and 6).

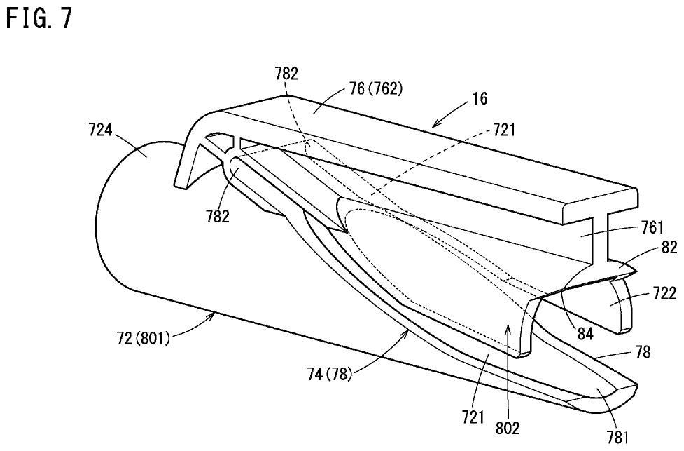

As illustrated in FIG. 7, the needle protection member 16 has a cover portion 72, a patch accommodating portion 74, and a connection portion 76.

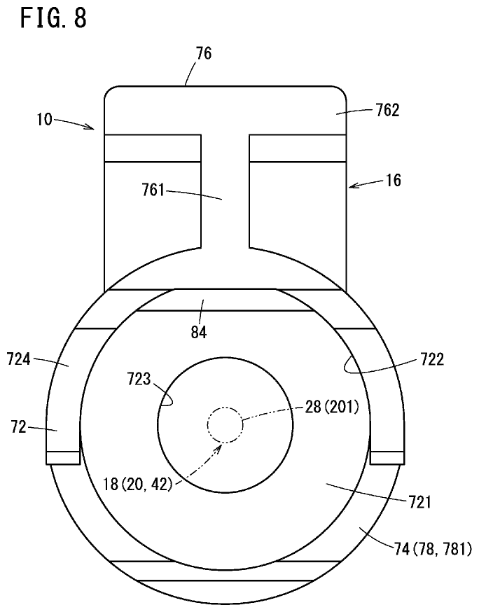

The cover portion 72 has a cylindrical shape having an accommodation space 721 inside and extending in an axial direction. When the needle protection member 16 is disposed so as to cover the outer periphery of the catheter 28, the outer periphery of the catheter 28 is covered by the cover portion 72. As illustrated in FIG. 4, a proximal end of the cover portion 72 has an opening 722. The opening 722 is continuous with and communicates with the accommodation space 721. As illustrated in FIG. 8, when the catheter 28 is accommodated inside the cover portion 72, an inner diameter of the opening 722 is larger than a diameter (outer diameter) of the catheter 28 when viewed from an axial direction of the catheter 28. An outer peripheral surface of the catheter 28 and the opening 722 are spaced apart in a radial direction. The outer peripheral surface of the catheter 28 and an inner peripheral surface of the cover portion 72 are spaced apart in the radial direction. In a state in which the catheter 28 is accommodated in the accommodation space 721 of the cover portion 72, the cover portion 72 and the catheter 28 are out of contact with each other. As illustrated in FIG. 3, a distal end of the cover portion 72 has a distal end opening 723. The distal end opening 723 has a smaller diameter than a diameter of the opening 722. The distal end opening 723 communicates with the accommodation space 721.

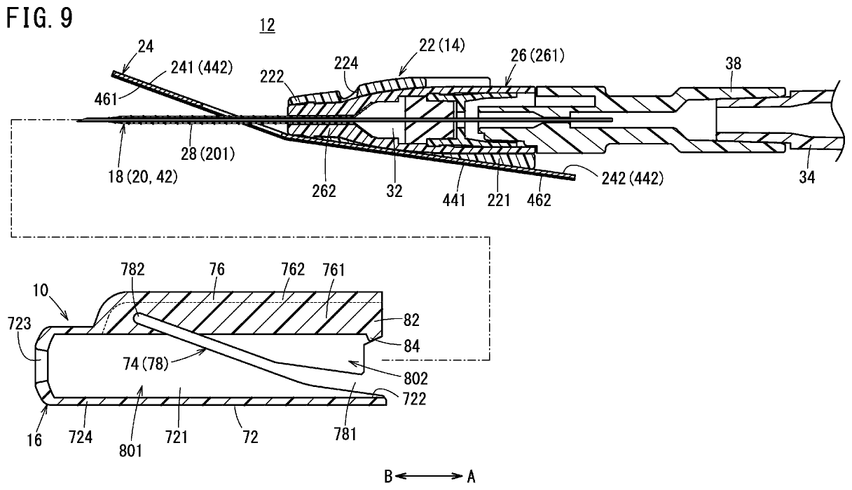

As illustrated in FIG. 4, at least a part of the patch 24 is accommodated in the patch accommodating portion 74 when the catheter 28 is accommodated in the cover portion 72. As illustrated in FIG. 7, the patch accommodating portion 74 has a pair of slit portions 78 that are provided in the cover portion 72 and intersect an axial direction of the cover portion 72. The pair of slit portions 78 face each other with across an axis of the cover portion 72. As illustrated in FIG. 9, the slit portions 78 intersect a peripheral wall 724 of the cover portion 72. The slit portions 78 are inclined with respect to the axial direction of the cover portion 72 and penetrate a part of the peripheral wall 724 of the cover portion 72. As illustrated in FIG. 4, in a state in which the needle protection member 16 is attached to the needle unit 14, the slit portions 78 extend upward from below the cover portion 72. The slit portions 78 are inclined upward toward a distal direction. An inclination angle of the slit portions 78 with respect to the axial direction of the cover portion 72 is, for example, about 30 degrees. The inclination angle of the slit portions 78 is not limited to 30 degrees. For example, the inclination angle of the slit portions 78 may be within a range of 5 to 45 degrees.

As illustrated in FIG. 9, each slit portion 78 includes an open end portion 781 and a closed end portion 782 provided on a side opposite to the open end portion 781. The open end portion 781 opens to an outside of the cover portion 72. The open end portion 781 is provided at one end portion of the slit portion 78 and extends to the opening 722 of the cover portion 72. The open end portion 781 is an insertion portion when the patch 24 is accommodated in the patch accommodating portion 74. The closed end portion 782 is provided at the other end portion (distal end portion) of the slit portion 78. The closed end portion 782 is disposed on a distal direction side of the cover portion 72 with respect to the open end portion 781. The closed end portion 782 is provided in the connection portion 76. The slit portion 78 extends upward from the open end portion 781 toward the closed end portion 782. The open end portion 781 of the slit portion 78 is not limited to a case in which the open end portion 781 is provided at the opening 722 of the cover portion 72. For example, the open end portion 781 of the slit portion 78 may be disposed in a distal direction (direction of arrow B) with respect to the opening 722 of the cover portion 72.

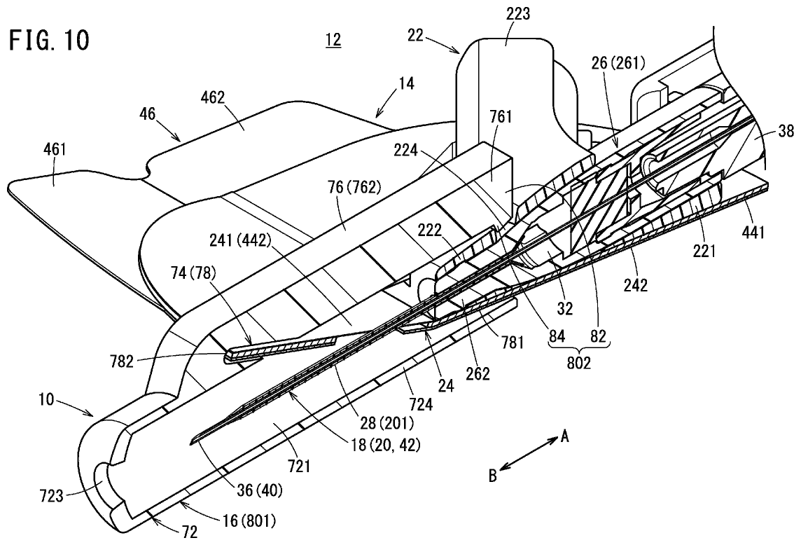

As illustrated in FIG. 10, the patch 24 is accommodated in the patch accommodating portion 74, the first patch portion 241 of the patch 24 first, through the open end portion 781. In the patch accommodating portion 74, a distal end portion of the patch 24 faces the closed end portion 782. An intermediate portion between the distal end portion and a proximal end portion of the patch 24 is disposed at the open end portion 781.

As illustrated in FIG. 4, when the needle protection member 16 is attached so as to cover the outer periphery of the catheter 28, the first patch portion 241 of the patch 24 is inserted into the patch accommodating portion 74 of the needle protection member 16 in a state inclined with respect to the second patch portion 242. When the needle protection member 16 is attached to the needle unit 14, the patch 24 accommodated in the patch accommodating portion 74 and a distal end portion of the catheter 28 are held in a spaced state. The patch 24 and the distal end portion of the catheter 28 are out of contact with each other. The patch 24 is exposed to an outside of the cover portion 72 through the pair of slit portions 78. In a width direction of the patch 24, the width direction being orthogonal to the axial direction of the needle protection member 16 and being an extending direction of the patch 24, the patch 24 protrudes to both sides in the width direction with respect to the peripheral wall 724 of the cover portion 72 (see FIG. 1). A central portion of the patch 24 in the width direction is accommodated in the accommodation space 721 of the needle protection member 16.

The patch accommodating portion 74 is not limited to a case in which the patch accommodating portion 74 is provided in the cover portion 72. For example, a patch accommodating portion 74 that protrudes outward in the width direction from the peripheral wall 724 of the cover portion 72 and is capable of accommodating the patch 24 may be provided.

As illustrated in FIG. 9, the cover portion 72 further includes a first region 801 and a second region 802 separated from the first region 801 by the slit portions 78. The first region 801 includes a distal end portion of the cover portion 72. The first region 801 is a region between the slit portions 78 and the distal end of the cover portion 72. The second region 802 includes at least a part of a proximal end portion of the cover portion 72. The second region 802 is a region between the slit portions 78 and the proximal end of the cover portion 72. A proximal end of the second region 802 includes an extension portion 82 protruding in the proximal direction, and an engaging portion 84 protruding radially inward from the extension portion 82. The extension portion 82 is a region in which a part of the proximal end of the second region 802 protrudes. As illustrated in FIG. 4, the engaging portion 84 protrudes from an inner peripheral surface of the peripheral wall 724 of the cover portion 72. When the needle protection member 16 is attached to the needle unit 14, a distal end portion of the hub holding part 222 of the needle hub 261 is inserted into the opening 722 of the cover portion 72. At this time, the extension portion 82 faces an upper surface of the hub holding part 222, and the engaging portion 84 is inserted into the hole 224 of the hub holding part 222. By engaging the engaging portion 84 with the hole 224, movement of the needle protection member 16 with respect to the holder 22 in the distal direction (direction of arrow B) is restricted in an initial state before use of the medicinal liquid administration device 12.

The connection portion 76 connects an upper part of the first region 801 and an upper part of the second region 802 on an outer surface of the cover portion 72. The connection portion 76 is provided on an outer periphery of the cover portion 72. The connection portion 76 is disposed parallel to the cover portion 72 along an outer peripheral surface of the cover portion 72.

As illustrated in FIG. 8, the connection portion 76 has a first wall portion 761 protruding from the outer peripheral surface of the cover portion 72, and a second wall portion 762 provided at an end portion of the first wall portion 761. When the needle protection member 16 is viewed from the axial direction, the connection portion 76 is formed in a T-shape by the first wall portion 761 and the second wall portion 762. The first wall portion 761 protrudes radially outward from the outer peripheral surface of the cover portion 72. As illustrated in FIG. 9, the first wall portion 761 connects the outer peripheral surface of the first region 801 and the outer peripheral surface of the second region 802. A part of the slit portion 78 is provided in the first wall portion 761. The second wall portion 762 intersects the first wall portion 761. The closed end portion 782 of the slit portion 78 is provided in the first wall portion 761 of the connection portion 76. The connection portion 76 is not limited to a T-shape including the first wall portion 761 and the second wall portion 762.

As illustrated in FIG. 5, the device main body 54 includes a case 86 that has a function of storing the medicinal liquid S and delivering the medicinal liquid S from a distal end portion at an appropriate timing, and that has an accommodation space (not illustrated) formed therein for accommodating the respective components.

The case 86 includes therein a prefilled syringe 56 that stores the medicinal liquid S, a movement mechanism 90 that moves a first gasket 88 in the prefilled syringe 56, a control unit 92 that performs drive control of the movement mechanism 90, and a power supply unit (not illustrated) capable of supplying electric power to the respective components.

The prefilled syringe 56 is formed in a cylindrical shape along an axial direction (directions of arrows A and B), and is to be filled with the medicinal liquid S. A distal end portion of the prefilled syringe 56 protrudes from a distal end of the case 86 a predetermined length in the axial direction (direction of arrow B) and is exposed to the outside.

A distal end portion of the prefilled syringe 56 includes a delivery nozzle portion 94. By a packing (not illustrated) disposed in the delivery nozzle portion 94, the medicinal liquid S is held in a sealed state inside the prefilled syringe 56.

Next, an assembly process for attaching the needle protection member 16 to the needle unit 14 of the medicinal liquid administration device 12 will be described.

For example, in a state in which a needle tip of the needle unit 14 illustrated in FIG. 9 and the opening 722 of the needle protection member 16 face each other, after the catheter 28 is inserted, the needle tip first, into the opening 722 of the needle protection member 16, the needle protection member 16 is moved toward the needle unit 14. As a result, as illustrated in FIG. 4, the catheter 28 is accommodated in the accommodation space 721 of the needle protection member 16 from the distal end toward the proximal end. At this time, the first patch portion 241 is inserted from the open end portion 781 of the slit portion 78 of the patch accommodating portion 74. The catheter 28 is accommodated in the accommodation space 721 of the cover portion 72, and a part of the patch 24 is accommodated in the patch accommodating portion 74 along the slit portion 78. In the patch accommodating portion 74, the first patch portion 241 is accommodated closer to the closed end portion 782, and a part of the second patch portion 242 is accommodated closer to the open end portion 781.

By inserting the engaging portion 84 of the needle protection member 16 into the hole 224 of the hub holding part 222 in the needle hub 261 and engaging the engaging portion 84 with the hole 224, the needle protection member 16 is supported with respect to the holder 22 in a state in which a proximal end of the cover portion 72 covers the distal end of the hub holding part 222. In an initial state before use of the medicinal liquid administration device 12, movement of the needle protection member 16 with respect to the catheter 28 in the distal direction (direction of arrow B) is restricted. In the accommodation space 721, the catheter 28 is accommodated inside the cover portion 72 in a state in which the outer peripheral surface of the catheter 28 and the inner peripheral surface of the cover portion 72 are spaced apart and have a sufficient gap. That is, the catheter 28 is accommodated in the accommodation space 721 of the needle protection member 16 in a state in which the catheter 28 and the needle protection member 16 are out of contact with each other. A distal end of the catheter 28 and a distal end of the cover portion 72 are spaced apart in the axial direction.

As a result, the assembly process of the medical puncture needle 10 for attaching the needle protection member 16 to the needle unit 14 is completed in a state in which the needle protection member 16 covers the outer periphery of the catheter 28 and protects the catheter 28.

Next, a case in which the medicinal liquid S is administered to the user P using the medicinal liquid administration device 12 will be described. In use of the medicinal liquid administration device 12, the catheter 28 is indwelled through the body surface P1 of the user P, and, after a priming liquid delivery tool not illustrated is connected and an interior of the catheter 28 is filled with priming liquid, the device main body 54 is connected to perform automatic administration of the medicinal liquid S to the user P.

First, a first priming operation is performed to fill interiors of the catheter 28 and the tube 30 with priming liquid.

A priming liquid delivery tool is connected to a proximal end of the second connection member 68 with respect to the connector 48 of the medicinal liquid administration device 12 illustrated in FIG. 2. Priming liquid flows into an interior of the second connection member 68. The priming liquid flows to the tube 30 through the connector 48. The priming liquid flows from the tube 30 through the side port 263 to the space 32 of the needle hub 261. As a result, interiors of the tube 30, the space 32, and the catheter 28 are filled with the priming liquid, and the first priming operation is completed. When supply of the priming liquid by the priming liquid delivery tool is stopped, communication between the first connection member 66 and the tube 30 is shut off by a valve body not illustrated.

Next, the catheter 28 is indwelled in the body of the user P. After the user P grips the catheter 28 and positions the catheter 28 at a desired position on the body surface P1, the needle protection member 16 is removed from the catheter 28 toward the distal direction.

After the user P causes the engaging portion 84 of the needle protection member 16 to disengage from the hole 224 of the holder 22 and releases an engaged state, the user P moves the needle protection member 16 with respect to the needle unit 14 in the distal direction (direction of arrow B) along the axial direction, as illustrated in FIG. 9. When a proximal end of the cover portion 72 reaches a distal end of the inner needle 36, the needle protection member 16 is withdrawn from the catheter 28 and the inner needle 36.

As illustrated in FIG. 11, after the user P punctures the body with a distal end of the catheter 28 including the catheter 28 and the inner needle 36, the user P peels the first sheet portion 461 from the first patch portion 241 and attaches a part of the patch 24 along the body surface P1 of the user P by the first patch portion 241. As a result, the distal end of the catheter 28 is fixed to the body surface P1 in a state in which the distal end of the catheter 28 punctures the user P.

Next, by pulling the needle holding part 38 and the gripping member 34 with respect to the catheter 28 in the proximal direction (direction of arrow A), the inner needle 36 held by the needle holding part 38 is withdrawn from the needle hub 261. At this time, because the catheter 28 is fixed to the body surface P1, the inner needle 36 is removed from an interior of the catheter 28, and only the catheter 28 is indwelled in the body.

After indwelling of the catheter 28 is completed, a second priming operation is performed to fill interiors of the catheter 28 and the tube 30 again with priming liquid.

The second priming operation is performed using the priming liquid remaining in the priming liquid delivery tool used in the first priming operation. After the second priming operation is completed, the priming liquid delivery tool is removed together with the second connection member 68. The second priming operation is performed after the second sheet portion 462 is peeled off from the second patch portion 242 of the patch 24 and the second patch portion 242 is attached along the body surface P1 of the user P.

After the user P releases connection of the second connection member 68 to the first connection member 66, the user P pulls out the priming liquid delivery tool together with the second connection member 68 toward the proximal direction (direction of arrow A) and removes the priming liquid delivery tool.

Next, as illustrated in FIG. 5, the device main body 54 is attached to the connector 48 to administer the medicinal liquid S.

A distal end portion of the prefilled syringe 56 of the device main body 54 is inserted into an interior of a proximal end of the first connection member 66 in the connector 48. The hollow needle 64 punctures an axial center of a packing not illustrated. A distal end portion of the device main body 54 is connected to a proximal end of the connector 48.

After the user P turns on a power switch 96 provided in the case 86 to start the device main body 54, the control unit 92 measures timing for start of administration of the medicinal liquid by the medicinal liquid administration device 12. When an administration start timing arrives, the movement mechanism 90 is driven by a control signal from the control unit 92, the first gasket 88 moves toward the distal direction, and the medicinal liquid S in the prefilled syringe 56 is pushed out toward the delivery nozzle portion 94. The medicinal liquid S flows into an interior of the first connection member 66 through the hollow needle 64. A valve body (not illustrated) is elastically deformed toward the distal direction by pressure of the medicinal liquid S, and, after the medicinal liquid S flows through the tube 30 to the space 32 of the needle hub 261, the medicinal liquid S is administered into the body of the user P through the lumen 181 of the catheter 28.

When administration of the medicinal liquid S from the device main body 54 into the body is completed and flow of the medicinal liquid S stops, the valve body returns to an original shape by elasticity. As a result, administration of the medicinal liquid S into the body of the user P is completed.

The first embodiment provides the following effects.

As illustrated in FIG. 3, the medical puncture needle 10 includes the needle unit 14 having the catheter 28 (needle 18), and the needle protection member 16 that protects the catheter 28 by covering an outer periphery of the catheter 28 and is removable from the needle unit 14 when the catheter 28 is used. The needle protection member 16 has the accommodation space 721 in which the catheter 28 is accommodated, and includes the tubular cover portion 72 extending in the axial direction to cover the outer periphery of the catheter 28. In a state in which the catheter 28 is accommodated in the cover portion 72, the cover portion 72 and the catheter 28 are provided out of contact with each other. As a result, when the needle protection member 16 is attached to the needle unit 14 in the assembly process of the medical puncture needle 10, contact of a distal end portion of the catheter 28 with the opening 722 of the cover portion 72 can be avoided. Therefore, damage to the distal end portion of the catheter 28 due to contact with the proximal end of the cover portion 72 can be suppressed. In addition, in an initial state of the medicinal liquid administration device 12 in which the catheter 28 is accommodated in the accommodation space 721 of the needle protection member 16, the needle protection member 16 and the catheter 28 are out of contact with each other, and therefore damage to the distal end portion of the catheter 28 due to contact with the cover portion 72 can be suppressed.

The catheter 28 is a flexible catheter, and the medical puncture needle 10 has the inner needle 36 inserted through the catheter 28. As a result, when the medical puncture needle 10 is assembled, the distal end portion of the flexible catheter 28 can be effectively protected. Because the needle protection member 16 is supported by the holder 22, the needle protection member 16 can be supported with a simple configuration.

The needle member 20 is the outer needle member 201, and the needle unit 14 includes the inner needle member 40 having the inner needle 36 inserted into the lumen 181 of the catheter 28 and the needle holding part 38 holding a proximal end of the inner needle 36, and the inner needle member 40 is attached to the outer needle member 201 so as to be removable. As a result, in the assembly process of the medical puncture needle 10 including the needle unit 14 having the double needle 42, damage to the distal end portion of the catheter 28 forming an outer side of the double needle 42 can be effectively suppressed.

As illustrated in FIG. 4, the needle protection member 16 has the patch accommodating portion 74 that accommodates at least a part of the patch 24. As a result, deformation, damage, and the like of the patch 24 before use can be effectively suppressed by protecting the catheter 28 with the needle protection member 16 and accommodating the patch 24 in the patch accommodating portion 74.

The needle protection member 16 holds the distal end portion of the catheter 28 and the patch 24 in a state in which the distal end portion of the catheter 28 and the patch 24 are spaced apart from each other. As a result, contact of the distal end portion of the catheter 28 with the patch 24 is prevented by the needle protection member 16, and therefore the distal end portion of the catheter 28 can be kept clean.

The patch accommodating portion 74 is the slit portion 78 provided in the cover portion 72 of the needle protection member 16 and intersecting an inner wall of the cover portion 72. As a result, in the needle protection member 16, accommodation of the catheter 28 in the accommodation space 721 of the cover portion 72 and accommodation of the patch 24 in the patch accommodating portion 74 by the slit portion 78 can be performed simultaneously.

The cover portion 72 has the connection portion 76 connecting the first region 801 and the second region 802 on an outer surface of the cover portion 72. As a result, a large accommodation range of the patch 24 can be secured by the slit portion 78, and a suitable size of the patch 24 can be secured.

The slit portion 78 extends from the opening 722 to the connection portion 76. As a result, by enlarging a range of the slit portion 78, a larger accommodation range of the patch 24 can be secured and a large patch 24 can be accommodated.

By engaging the engaging portion 84 of the needle protection member 16 with the hole 224 of the hub holding part 222 of the holder 22, coming off of the needle protection member 16 in the distal direction can be effectively prevented in a state in which the needle protection member 16 is attached to the needle unit 14.

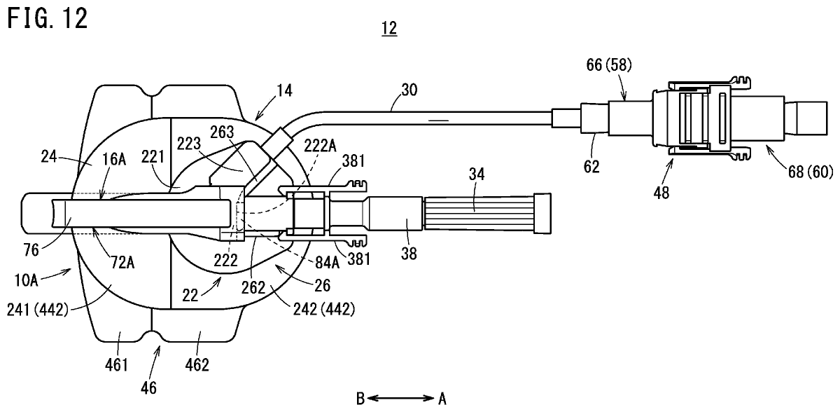

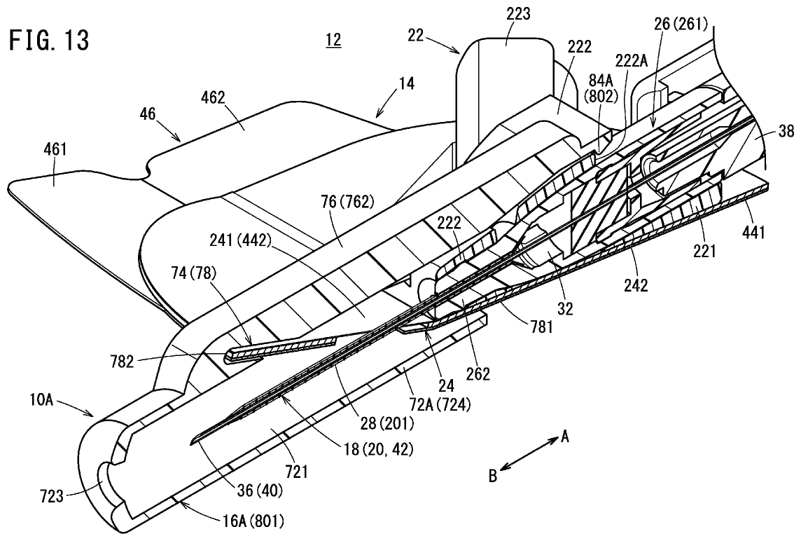

A medical puncture needle 10A according to a modification illustrated in FIG. 12 has a needle protection member 16A supported by the holder 22. As illustrated in FIG. 13, a cover portion 72A of the needle protection member 16A has the first region 801, the second region 802, and the patch accommodating portion 74. In a state in which the needle protection member 16A is attached to the needle unit 14, a proximal end portion of the second region 802 covers parts of the hub holding part 222 and the port holding part 223 of the holder 22. A proximal end of the second region 802 has an engaging portion 84A protruding radially inward from an inner peripheral surface of the proximal end. When the needle protection member 16A is attached to the needle unit 14, the engaging portion 84A is engaged with a proximal end 222A of the hub holding part 222. The proximal end 222A of the hub holding part 222 is an end portion orthogonal to an axial direction of the needle hub 261. By engaging the engaging portion 84A with the proximal end 222A of the hub holding part 222, movement of the needle protection member 16A in the distal direction with respect to the holder 22 is prevented.

When the needle protection member 16A is removed from the needle unit 14, after an engaged state is released by causing the engaging portion 84A of the needle protection member 16A to disengage from the proximal end 222A of the hub holding part 222, the needle protection member 16A is moved with respect to the needle unit 14 in the distal direction along the axial direction. When a proximal end of the cover portion 72A reaches a distal end of the inner needle 36, the needle protection member 16A is withdrawn from the catheter 28 and the inner needle 36.

The modification provides the following effects.

As illustrated in FIG. 13, because the needle protection member 16A is supported by the holder 22, the needle protection member 16A can be supported with a simple configuration. By engaging the engaging portion 84A of the needle protection member 16A with the proximal end 222A of the hub holding part 222 of the holder 22, coming off of the needle protection member 16A in the distal direction can be effectively prevented in a state in which the needle protection member 16A is attached to the needle unit 14.

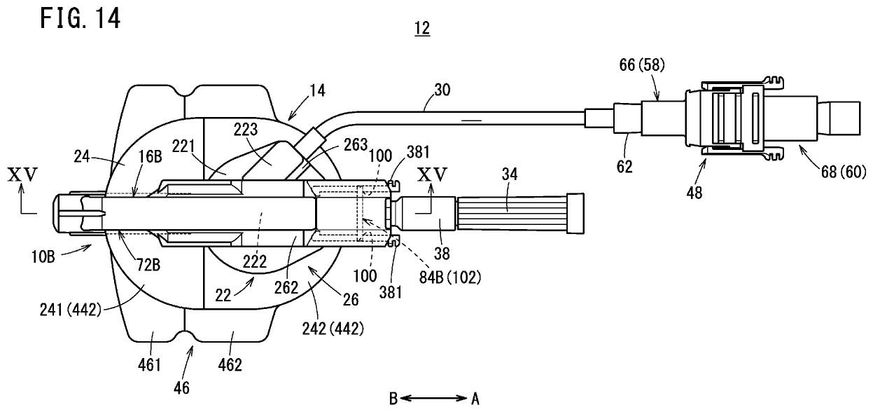

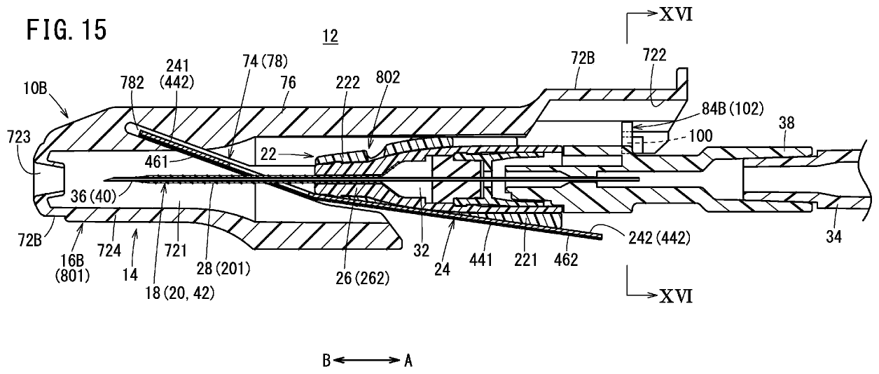

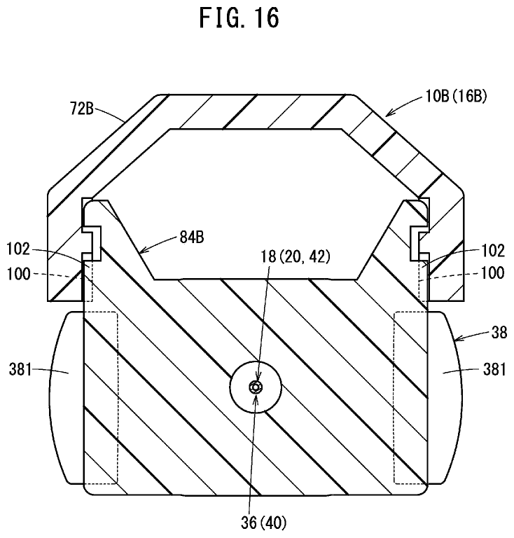

The medical puncture needle 10B according to the second embodiment illustrated in FIG. 14 has a needle protection member 16B supported by the needle holding part 38. As illustrated in FIG. 15, a cover portion 72B of the needle protection member 16B has the first region 801, the second region 802, and the patch accommodating portion 74. In a state in which the needle protection member 16B is attached to the needle unit 14, a proximal end portion of the second region 802 covers a distal end portion of the needle holding part 38. A proximal end portion of the second region 802 has an engaging portion 84B on an inner surface facing the needle holding part 38. As illustrated in FIG. 16, the engaging portion 84B has a pair of recesses 100 provided in a width direction orthogonal to an axial direction of the needle holding part 38. The pair of recesses 100 are recessed outward in the width direction on an inner surface of the cover portion 72B.

The needle holding part 38 has a protrusion 102 protruding upward from the pressing portions 381. The protrusion 102 is accommodated inside the cover portion 72B. In a state in which the needle protection member 16B is attached to the needle unit 14, a part of the protrusion 102 is engaged with the recesses 100. By the engaging portion 84B being engaged with the recesses 100 of the cover portion 72B, movement of the needle protection member 16B in the distal direction with respect to the holder 22 is restricted in an initial state before use of the medicinal liquid administration device 12.

When the needle protection member 16B is removed from the needle unit 14, by pulling the needle protection member 16B in the distal direction with respect to the needle unit 14, the recesses 100 disengage from the protrusion 102 and an engaged state of the engaging portion 84B is released. When a proximal end of the cover portion 72B reaches a distal end of the inner needle 36, the needle protection member 16B is withdrawn from the catheter 28 and the inner needle 36.

The second embodiment provides the following effects.

As illustrated in FIG. 16, because the needle protection member 16B is supported by the protrusion 102 of the needle holding part 38, the needle protection member 16B can be supported with a simple configuration in a state in which the catheter 28 is accommodated. By engaging the engaging portion 84B (recesses 100) of the needle protection member 16B with the protrusion 102 provided at a proximal end portion of the needle holding part 38, coming off of the needle protection member 16B in the distal direction can be effectively restricted in an initial state before use of the medicinal liquid administration device 12 in a state in which the needle protection member 16B is attached to the needle unit 14.

The present invention is not limited to the above-described disclosure, and various configurations may be adopted without departing from the gist of the present invention.

1. A medical puncture needle comprising:
a needle unit comprising:
a needle member comprising a needle that has a lumen,
a holder holding a proximal end of the needle member, and
a patch having a first surface configured to be attached to a body surface of a living body, and a second surface opposite to the first surface, at least a part of the second surface being connected to the holder; and
a needle protection member that covers an outer periphery of the needle and is removable from the needle unit when the needle is used, the needle protection member comprising:
a cover portion having a tubular shape and having an accommodation space in which the needle is accommodated, the cover portion extending in an axial direction to cover the outer periphery of the needle, and
an opening located at a proximal end of the cover portion and continuous with the accommodation space; wherein:
when viewed from an axial direction of the needle, the opening is larger than the needle; and
in a state in which the needle is accommodated in the cover portion, the cover portion and the needle are out of contact with each other.

2. The medical puncture needle according to claim 1, wherein:
the needle is a catheter having flexibility; and
the medical puncture needle comprises an inner needle inserted through the catheter.

3. The medical puncture needle according to claim 1, wherein:
the needle protection member is supported by the holder.

4. The medical puncture needle according to claim 1, wherein:
the needle member is an outer needle member;
the needle unit comprises an inner needle member that comprises:
an inner needle inserted into the lumen of the needle, and
a needle holding part holding a proximal end of the inner needle; and
the inner needle member is detachably attached to the outer needle member.

5. The medical puncture needle according to claim 4, wherein:
the needle protection member is supported by the holder or the needle holding part.

6. The medical puncture needle according to claim 1, wherein:
the needle protection member comprises a patch accommodating portion that accommodates at least a part of the patch.

7. The medical puncture needle according to claim 6, wherein:
the needle protection member holds a distal end of the needle and the patch in a state in which the distal end of the needle and the patch are spaced apart from each other.

8. The medical puncture needle according to claim 6, wherein:
the patch accommodating portion is a slit portion provided in the cover portion of the needle protection member and intersecting an inner wall of the cover portion.

9. The medical puncture needle according to claim 8, wherein:
the cover portion includes:
a first region including a distal end portion of the cover portion, and
a second region including at least a part of a proximal end portion of the cover portion and separated from the first region by the slit portion; and
the needle protection member comprises a connection portion connecting the first region and the second region on an outer surface of the cover portion.

10. The medical puncture needle according to claim 9, wherein:
the slit portion extends from the opening to the connection portion.

11. A medical puncture needle comprising:
an assembly comprising:
a catheter member comprising a catheter that has a lumen,
a holder holding a proximal end of the catheter member, and
a patch having a first surface configured to be attached to a body surface of a living body, and a second surface opposite to the first surface, at least a part of the second surface being connected to the holder; and
a protection member that covers an outer periphery of the catheter and is removable from the assembly when the catheter is used, the protection member comprising:
a cover portion having a tubular shape and having an accommodation space in which the catheter is accommodated, the cover portion extending in an axial direction to cover the outer periphery of the catheter, and
an opening located at a proximal end of the cover portion and continuous with the accommodation space; wherein:
when viewed from an axial direction of the catheter, the opening is larger than the catheter; and
in a state in which the catheter is accommodated in the cover portion, the cover portion and the catheter are out of contact with each other.

12. The medical puncture needle according to claim 11, further comprising:
an inner needle inserted through the catheter.

13. The medical puncture needle according to claim 11, wherein:
the protection member is supported by the holder.

14. The medical puncture needle according to claim 11, wherein:
the assembly comprises an inner needle member that comprises:
an inner needle inserted into the lumen of the catheter, and
a needle holding part holding a proximal end of the inner needle; and
the inner needle member is detachably attached to the catheter member.

15. The medical puncture needle according to claim 14, wherein:
the protection member is supported by the holder or the needle holding part.

16. The medical puncture needle according to claim 11, wherein:
the protection member comprises a patch accommodating portion that accommodates at least a part of the patch.

17. The medical puncture needle according to claim 16, wherein:
the protection member holds a distal end of the catheter and the patch in a state in which the distal end of the catheter and the patch are spaced apart from each other.

18. The medical puncture needle according to claim 16, wherein:
the patch accommodating portion is a slit portion provided in the cover portion of the protection member and intersecting an inner wall of the cover portion.

19. The medical puncture needle according to claim 18, wherein:
the cover portion includes:
a first region including a distal end portion of the cover portion, and
a second region including at least a part of a proximal end portion of the cover portion and separated from the first region by the slit portion; and
the protection member comprises a connection portion connecting the first region and the second region on an outer surface of the cover portion.

20. The medical puncture needle according to claim 19, wherein:
the slit portion extends from the opening to the connection portion.
